(19) United States
(12) Patent Application Publication
(10) Pub. No.: US 2022/0330707 A1
(43) Pub. Date: Oct. 20, 2022

(54) Vibrating Bed for Alerting Quiescent and Sleeping Individuals

(71) Applicant: Marshall Burgess, JR., Sparks, NV (US)

(72) Inventor: Marshall Burgess, JR., Sparks, NV (US)

(21) Appl. No.: 17/684,714

(22) Filed: Mar. 2, 2022

Related U.S. Application Data
(60) Provisional application No. 63/176,554, filed on Apr. 19, 2021.

(51) Int. Cl. A47C 21/00; G08B 17/10; G08B 1/08; G08B 6/00; A61M 21/00; G08B 3/10

(52) U.S. Cl. CPC A47C 21/006; G08B 17/10; G08B 1/08; G08B 6/00; A61M 21/00; G08B 3/10; A61M 2021/0022; A61M 2021/0083

(57) ABSTRACT

The present invention relates to a system, method and a novel vibrating bed to alert and wake up sleeping and quiescent individuals when a fire occurs. The vibrating bed includes a vibrating mechanism that includes a vibrating motor to shake and vibrate rubber tubes to shake the bed to alert and wake up individuals when smoke or fire is detected by a smoke/fire detector. The smoke/fire detector is wirelessly coupled to the vibrating bed through a wireless communication channel and a processor of the bed receives an alert signal to actuate the vibrating motor from the detector when the smoke/fire detector detects fire or smoke.

### CROSS-REFERENCE TO RELATED APPLICATION

The present application claims priority to, and the benefit of, U.S. Provisional Application No. 63/176,554, which was filed on Apr. 19, 2021 and is incorporated herein by reference in its entirety.

### FIELD OF THE INVENTION

The present invention relates generally to smoke and fire alerting devices and systems. More specifically, the present invention relates to a smoke and fire alert bed mechanism and system that are designed to allow a fire/smoke detector to detect the presence of a smoke and/or fire to activate a vibration mechanism of the smoke and fire alert bed. The smoke and fire alert bed includes a vibration motor that shakes and vibrates rubber tubes which shake the bed and wakes up the person sleeping on the bed when the detector detects fire or smoke. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, smoke and fire detectors are lifesaving devices that greatly increase the likelihood of surviving a fire in a building. Generally, these detection devices sense smoke, fire, carbon dioxide, or other noxious and toxic airborne substances, and in response, sound a piercing alarm, and/or flash an intense strobe light, to alert the occupants of the building to the dangerous condition. For residential structures, in many scenarios, occupants can be sleeping and difficult to rouse. For example, young children often fail to wake during a fire, due in part, to their ability to fall asleep even in the noisiest environments. Older adults may also be insensitive to external ambient or environmental dangers when sleeping. However, standard smoke detectors' audible and visual alarms often fail to wake the individuals quickly enough to avoid escape and injury. Further, it takes a considerable amount of time for a fire department to respond to a fire and to protect and take individuals out from the fire. Critical time can be wasted or generally passed due to the ineffective alerting of sleeping people.

Inefficiency or ineffectiveness of alerting people by heretofore known standard detectors is borne out by statistics that show thousands of people die from residential fires and smoke inhalation every year. In fact, most fire deaths are not caused by burns, but by inhaling deadly smoke and fumes generated by the fire.

Sleeping and quiescent people generally can get up quickly and escape from the fire and smoke if there is a means of quickly waking them up. Remote help such as through calls, neighbors, good Samaritans, and/or messages is inefficient.

Therefore, there exists a long felt need in the art for an improved fire and smoke alerting device that can be used for immediately alerting and awakening quiescent and sleeping people. There is also a long felt need in the art for an improved fire and smoke alerting device and system that allows users to quickly escape fire and smoke. Additionally, there is a long felt need in the art for an improved bed that can vibrate to alert and awake sleeping individuals. Moreover, there is a long felt need in the art for an improved fire and smoke alerting device that is useful for adults, children, and pets. Furthermore, there is a long felt need in the art for an improved fire and smoke alerting device that improves the efficiency of the standard detectors and can work with them to immediately alert users. Finally, there is a long felt need in the art for an improved fire and smoke alerting device that enables a way for individuals to quickly escape from the fire and smoke.

The subject matter disclosed and claimed herein, in one embodiment thereof, comprises a vibrating bed that automatically vibrates in response to fire or smoke detected by a fire and smoke alarm for alerting and waking quiescent people. The vibrating bed is communicatively coupled to a home fire and smoke alarm through a wireless channel. The vibrating bed further includes a vibration motor, a plurality of rubber tubes configured to vibrate and shake by the vibration motor, the vibration motor is actuated to vibrate and shake the rubber tubes when a processor of the bed receives a wireless alert signal from the room, home, or building fire and smoke alarm, the alarm transmits the wireless alert signal upon detecting smoke or fire, and the rubber tubes vibrate and shake the users lying on the bed in order to wake them up. The bed also contains a weight sensor for sensing weight on the bed, wherein if no weight is detected on the bed, then, no vibration is performed in the bed.

In this manner, the vibrating or fire protection bed of the present invention accomplishes all of the forgoing objectives and provides users with a bed connected to a fire alarm via a wireless connection and allows the alarm to detect the presence of a smoke and/or fire to activate a vibration mechanism within the bed. The vibrating motor and the tubes shake the bed in order to wake up the person(s) sleeping thereon to ensure that they can quickly escape the fire or smoke. The bed improves overall home safety to enable adults, children and pets to escape their home should a fire occur.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a system for automatically vibrating a bed in response to fire or smoke detected by a fire and smoke alarm for alerting and waking quiescent people. The system further comprises a vibrating bed communicatively coupled to a home fire and smoke alarm through a wireless channel, the vibrating bed includes a vibration motor, a plurality of rubber tubes configured to vibrate and shake by the vibration motor, the vibration motor is actuated to vibrate and shake the rubber tubes when a processor of the bed receives a wireless alert signal from the room, home or building fire and smoke alarm, the alarm transmits the wireless alert signal upon detecting smoke or fire, the rubber tubes vibrate and shake the user(s) lying on the bed to wake them up. The bed also contains a weight sensor for sensing weight on the bed wherein if no weight is detected on the bed, then no vibration is performed in the bed.

In yet another embodiment, the vibration motor receives electrical power from a conventional 120-volt AC power outlet.

In yet another embodiment, the wireless channel is established by a wireless module of the vibrating bed and the wireless channel uses one of the Bluetooth, Wi-Fi, or any other short range communication channel.

In yet another embodiment, the vibrating bed can be a king bed, a queen bed, a California king bed, a single bed, a full bed or any other type of bed.

The subject matter disclosed and claimed herein, in another embodiment thereof, comprises a vibrating bed configuration. The vibrating bed configuration further comprises a skeleton structure with an integrated vibrating system. The vibrating system includes a vibration motor attached to the bottom surface of the skeleton structure; a USB box coupled to the vibration motor, the USB box storing a processor; a wireless module; a weight sensor; and a speaker; and a plurality of rubber tubes disposed on the bottom surface of the skeleton structure. The vibration motor is configured to vibrate and shake the rubber tubes to shake the skeleton structure wherein the vibration motor is automatically activated when the processor receives a wireless signal from a room, home, or building fire/smoke detector for smoke or fire detected by the room, home or building fire/smoke detector.

In yet another embodiment, the vibrating bed configuration includes an actuation button for deactivating the vibration motor.

In yet another embodiment, the vibrating system can be retrofitted to any conventional bed to transform the bed into the vibrating bed configuration.

In yet another embodiment of the present invention, a method for alerting one or more users sleeping on a vibrating bed in case fire or smoke is detected is described. The method includes wirelessly coupling the vibrating bed and fire or smoke detector, receiving a wireless instruction signal by a processor of the vibrating bed from the fire or smoke detector when fire or smoke is detected by the fire or smoke detector, actuating a vibration motor attached to the vibrating bed by the processor for shaking and vibrating rubber tubes disposed on the bottom surface of the vibrating bed wherein the continuous vibration alerts one or more users and wakes them up for protecting the users from fire and/or smoke.

In yet another embodiment, frequency of the vibrations is increased when the users do not wake up.

In yet another embodiment, a vibrating bed configuration associated with an Internet of Things (IoT) network is disclosed. The vibrating bed configuration is configured to couple with an IoT hub through a wireless communication channel wherein the bed receives an alert signal to vibrate the bed using vibrating rubber tubes to alert and awake users sleeping or lying on the bed. The IoT hub transmits an alert signal to the bed when fire or smoke is detected by any of the smart devices connected with the IoT hub.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of one potential embodiment of a fire and smoke alert or detector system of the present invention in accordance with the disclosed architecture;

FIG. 2 illustrates a block diagram showing components of an exemplary fire and smoke alarm or detector device that is used in conjunction with a fire protection bed of the present invention in accordance with the disclosed architecture;

FIG. 3 illustrates a perspective view showing the fire protection bed being placed in a room and paired with the exemplary fire and smoke detector device for vibrating the bed in accordance with the disclosed architecture;

FIG. 4 illustrates a bottom perspective view of the fire protection bed showing position of a USB box, a vibrating motor, and rubber tubes on the bottom surface of the bed in accordance with the disclosed architecture;

FIG. 5 illustrates a flow diagram showing exemplary steps in generating vibration in the vibrating bed for waking up users lying on the bed in accordance with the disclosed architecture;

FIG. 6 illustrates a flow diagram showing steps performed in increasing the frequency of vibration produced by the vibrating motor used in the vibrating bed of the present invention in accordance with the disclosed architecture; and

FIG. 7 illustrates another schematic view of the fire and smoke prevention system of the present invention with the fire protection bed in communication with an Internet of Things (IoT) hub in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It can be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments can be combined.

As noted above, there is a long felt need in the art for an improved fire and smoke alerting device that can be used for immediately alerting and awakening quiescent and sleeping people. There is also a long felt need in the art for an improved fire and smoke alerting device and system that allows users to quickly escape fire and smoke. Additionally, there is a long felt need in the art for an improved bed that can vibrate to alert and awake sleeping individuals. Moreover, there is a long felt need in the art for an improved fire and smoke alerting device that is useful for adults, children and pets. Furthermore, there is a long felt need in the art for an improved fire and smoke alerting device that improves the efficiency of the standard detectors and can work with them to immediately alert users. Finally, there is a long felt need in the art for an improved fire and smoke alerting device that enables a way for individuals to quickly escape from the fire and smoke.

The present invention, in one exemplary embodiment, is a novel vibrating bed configuration. The vibrating bed configuration features a skeleton structure with an integrating vibrating system. The vibrating system includes a vibration motor attached to the bottom surface of the skeleton structure, a USB box coupled to the vibration motor, the USB box storing a processor, a wireless module, a weight sensor and a speaker, and a plurality of rubber tubes disposed on the bottom surface of the skeleton structure. The vibration motor is configured to vibrate and shake the rubber tubes to shake the skeleton structure wherein the vibration motor is automatically activated when the processor receives a wireless signal from a room, home, or building fire/smoke detector for smoke or fire detected by the room, home, or building fire/smoke detector.

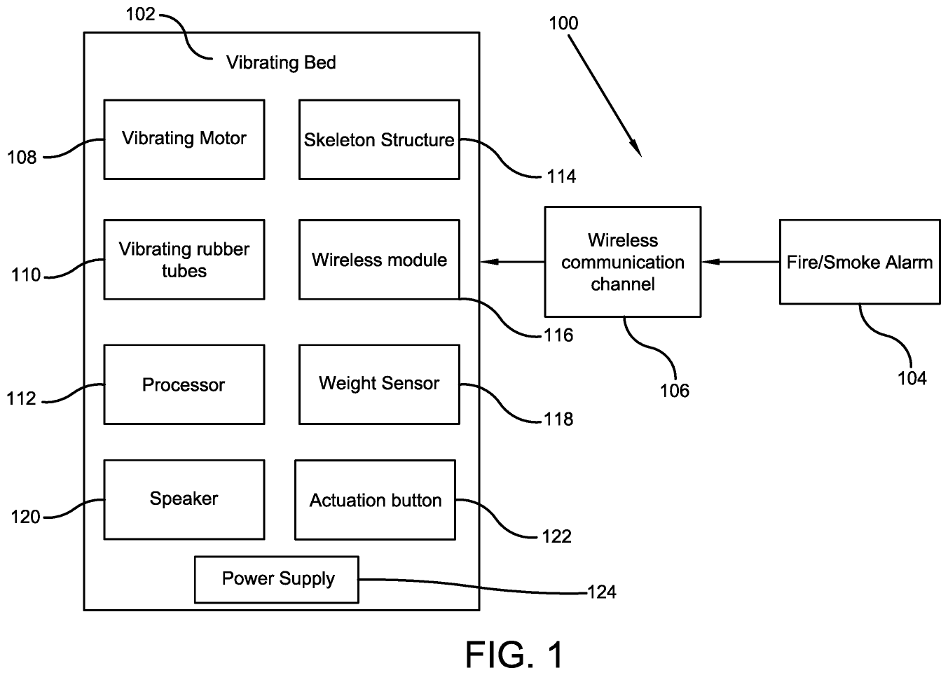

Referring initially to the drawings, FIG. 1 illustrates a schematic view of one potential embodiment of a fire and smoke alert system of the present invention in accordance with the disclosed architecture. FIG. 1 shows several advantageous features of the present invention, but as will be described below, the invention can be provided in several shapes, sizes, combinations of features and components, and varying numbers and functions of the components. The fire and smoke alert system 100 is designed using a novel vibrating bed 102 that can be in wireless communication with a room, home, or building fire or smoke alarm device 104 mounted at any appropriate place in the room where the vibrating bed 102 is placed. The bed 102 and the alarm device 104 are in wireless communication using a wireless communication channel 106, preferably Bluetooth. The vibrating bed 102 is configured to vibrate instantly to wake people sleeping on the bed 102 when the fire or smoke alarm device 104 detects fire or smoke. A wireless signal from the room, home, or building alarm device 104 is transmitted to the bed 102 when the smoke or fire is detected and that allows the bed 102 to vibrate to wake up the user. The wireless channel 106 can be of any local area network (LAN), metropolitan area network (MAN), wide area network (WAN), a public data network (e.g., the Internet), short range wireless network and the like, or any combination thereof. In addition, the wireless network can be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, mobile ad-hoc network (MANET) and the like, or any combination thereof.

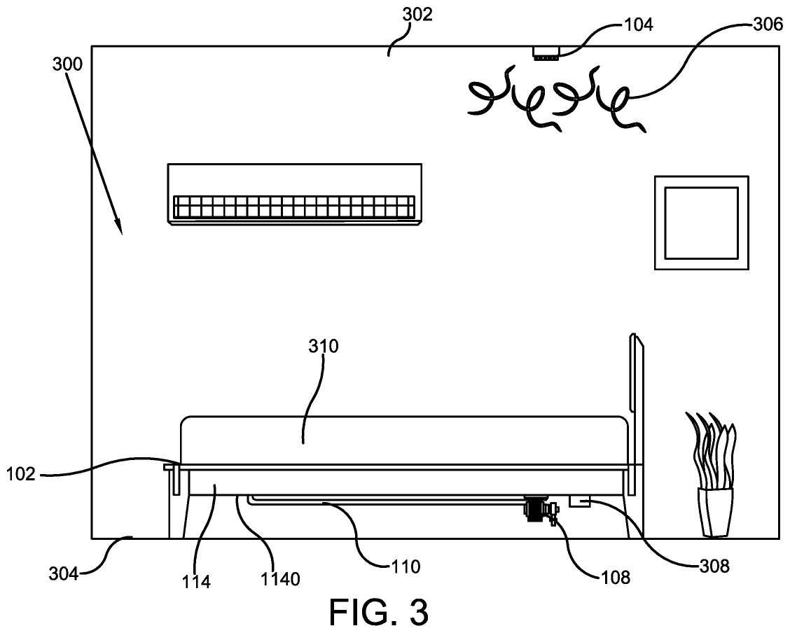

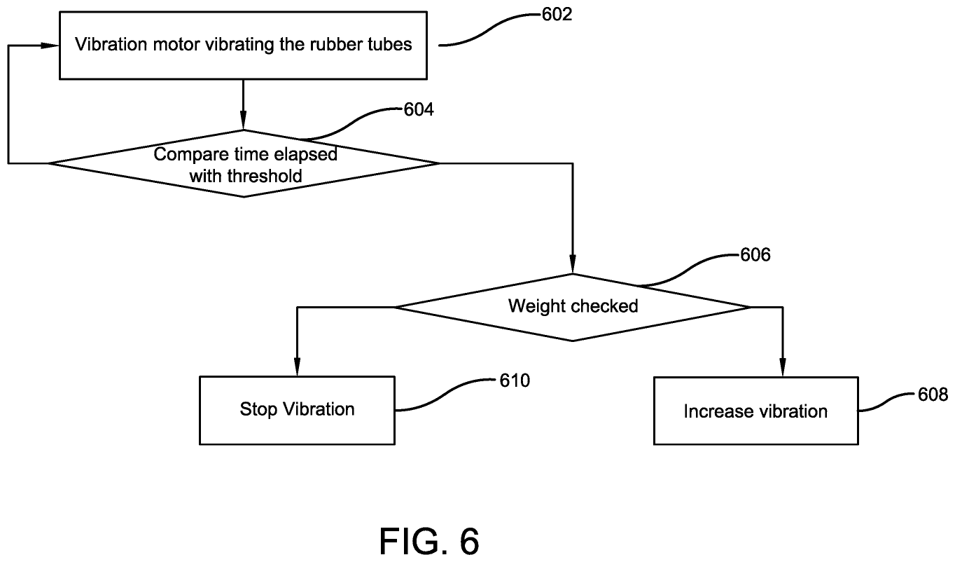

More specifically, the vibrating bed 102 includes a vibrating motor 108 that is disposed at a convenient location of the bed, preferably at the bottom surface of the skeleton structure 114 of the bed 102 as shown in FIG. 3. The vibrating motor 108 is activated when the processor 112 of the bed 102 received the wireless or alarm signal from the room, home or building fire alarm 104 and provides power to the vibrating rubber tubes 110 that are disposed throughout the bottom surface of the bed 102. The vibrating rubber tubes are configured to vibrate to create a sensation in and through the mattress to the users lying on the bed 102. The processor 112 is designed to control and operate the vibrating motor 108 and increase the vibrating frequency of the rubber tubes 110 in case the users lying on the bed 102 do not wake up and get off of the mattress when fire or smoke is detected as described best in FIG. 6. In addition to providing vibration for waking up the users, the bed 102 includes an integrated speaker 120 that produces an alarming sound when the fire or smoke wireless signal is received by the processor 112 from the alarm device 104.

For providing wireless communication capabilities to the vibration bed 102, a wireless module 116 is integrated. The wireless module 116 is used for establishing the wireless communication channel 106 between the bed 102 and the alarm device 104. According to embodiments of the present invention, the wireless module 116 can be in the form of an integrated chip and can be, but not limited to, a Bluetooth chip, a Wireless Fidelity (Wi-Fi) chip, an Infrared (IR) chip, Global System for Mobile Communication (GSM) chip, a Code-Division Multiple Access (CDMA) chip, a Near Field Communication (NFC) chip, and so forth. In a preferred embodiment of the present invention, the wireless device 116 is a Bluetooth Low Energy module.

For providing vibration only when at least one user is lying on the fire protection bed 102, a weight sensor 118 is disposed at the bottom surface of the bed. The weight sensor 118 can be optionally present in the bed 102 and is utilized to detect any weight on the bed. Further, the weight sensor 118 provides detected weight information to the processor 112 that is used for actuating the vibration motor 108. In situations where a weight is not detected by the weight sensor 118 on the bed, then it can be deduced that a user is not lying or sleeping on the fire protection bed 102 and thus vibration is not actuated. In this case, only speaker 120 can be activated to produce an alarming sound. It should be appreciated that the weight sensor 118 is an optional feature of the system and can be removed based on the preferences and desires of users. For manually deactivating the vibration motor 108, an actuation button is disposed on the bed 102 and can be pushed to deactivate the motor 108, based on the preferences of the users.

In use, the vibrating motor 108 shakes the rubber tubes 110 that shake the bed and the user(s) lying on the bed 102 in order to wake them up. For providing power to the motor 108, a power supply 124 is disposed in the bed 102. The power supply 124 can have a power cord that plugs into a conventional 120-volt AC power outlet, as is conventional in homes and other areas. Although, in the disclosure only one vibrating motor 108 is shown and described, it is to be appreciated that more than one motor can be used for providing vibration to the rubber tubes depending on the bed size and the power of vibration required by users.

It is to be appreciated that the vibrating motor 108, the vibrating rubber tubes 110, the processor 112, the wireless module 116, the weight sensor 118, the speaker 120 and the actuation button 122 are connected to each other through a wired circuit on the surface of the bed 102. Further, the processor 112, the wireless module 116, the weight sensor 118, and the speaker 120 can be formed as a single physical unit that houses these components. The components forming the vibrating bed 102 can be retrofitted to any existing bed or can be attached to a bed during manufacturing of the bed, thereby ensuring that users can quickly escape a dangerous situation due to fire or smoke.

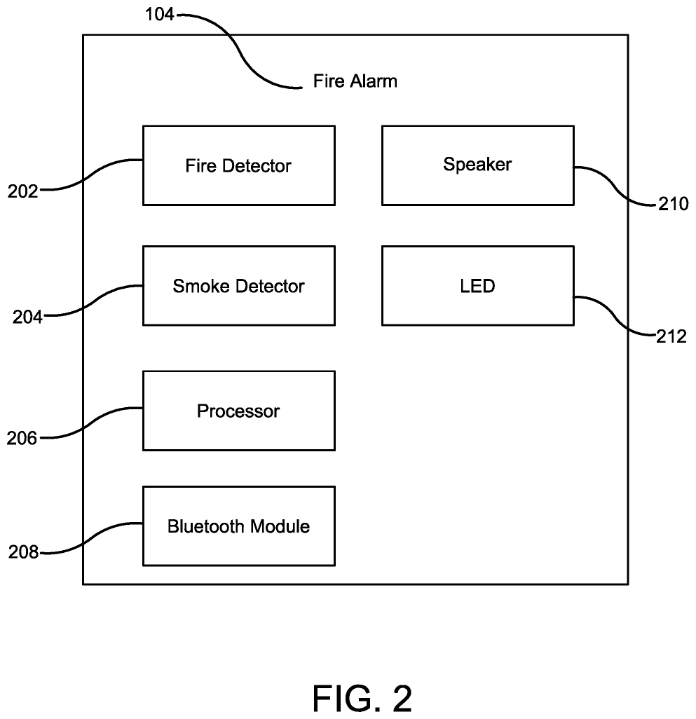

FIG. 2 illustrates a block diagram showing components of an exemplary fire and smoke alarm device 104 that is used in conjunction with the fire protection bed 102 of the present invention in accordance with the disclosed architecture. The fire and smoke detection and alarm device 104 can be an ionization detector or a photoelectric detector. The bed 102 can work with any type of fire and smoke detector 104. The alarm device 104 includes a fire detector 202 that detects fire by sensing small particles in the air. The fire detector component 202 can detect both slow-burning “smoldering” fires and fast-burning “flaming” fires. A smoke detector component 204 detects the smoke and the sensitivity of the smoke detection can be customized for effective protection from dangerous smoke and fire. A processor 206 of the alarm device 104 transmits a wireless signal to the bed 102 as shown in FIG. 1 when fire or smoke is detected by the alarm device 104 using the Bluetooth module 208. The Bluetooth module 208 establishes the wireless channel with the wireless module of the bed 102, thereby allowing transmission of the wireless signal from the processor 206. The alarm device 104 can have a speaker 210 for producing a sound for alerting the users in the room and in other surrounding areas indicating smoke or fire. For providing a visual alert, LEDs 212 are disposed on the fire alarm device 104 that can be flashed in a specific color such as “RED”, for example to indicate that a dangerous smoke or fire is detected by the device 104.

It should be appreciated that in one embodiment of the present invention, the fire and smoke alarm device 104 and the fire protection bed 102 come together in the form of a kit. Alternatively, the fire protection bed 102 can be paired with any conventional fire and smoke alarm device 104 wherein the fire and smoke alarm device 104 includes a wireless communication capability.

FIG. 3 illustrates a perspective view showing the fire protection bed 102 being placed in a room 300 and paired with the exemplary smoke and fire detector device 104 for vibrating the bed in accordance with the disclosed architecture. As shown, the vibrating bed 102 is placed on the floor 304 of the room 300 and the fire alarm 104 can be mounted to a wall or ceiling 302 of the room 300. At the bottom surface 1140 of the skeleton structure 114 of the bed 102, a USB box 308 is attached along with the vibrating motor 108. The USB box 308 collectively houses the processor 112, the wireless module 116, the weight sensor 118, and the speaker 120 as described in FIG. 1. The vibrating motor 108 is connected to the rubber tubes 110 that are disposed along the bottom surface 1140. The rubber tubes 120 can be shaken and vibrated by the vibrating motor 108. When the rubber tubes 110 are shaken, the mattress 310 placed on the bed 102 is shaken and vibrated and thus user(s) lying on the mattress 310 wake up instantly. In operation, when fire or smoke 306 is detected by the detector device 104, a wireless signal is transmitted to the processor 112 placed inside the USB box 308. The wireless signal is an indication that fire or smoke 306 is detected and accordingly, the processor 112 of the bed 102 actuates the motor 108 to shake and vibrate the rubber tubes 110.

The vibrating motor 108 is preferably in the form of a mini-motor that can have a weight in the range of about 0.5 kg to about 1 kg and vibrates at least in the range 10,000 rpm-15,000 rpm. The rubber tubes 110 directly shake and vibrate the mattress 310.

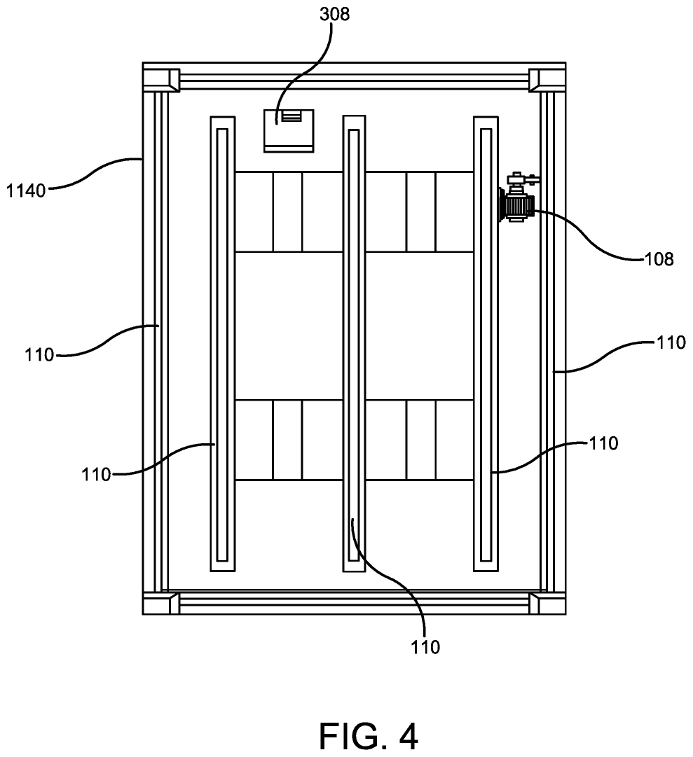

FIG. 4 illustrates a bottom perspective view of the fire protection bed 102 showing a position of the USB box 308, vibrating motor 108, and the rubber tubes 110 on the bottom surface 1140 of the bed 102 in accordance with the disclosed architecture. The vibrating system comprising the USB box 308, vibrating motor 108, and the rubber tubes 110 is preferably disposed to the bottom surface 1140 of the bed 102 with a wired circuit (not shown) connecting these components. In one embodiment, more than one vibrating motor can be disposed and connected to the rubber tubes 110 to shake and vibrate the bed 102. As shown, the rubber tubes 110 are distributed along the bottom surface 1140 and vibrate by receiving power from the vibrating motor 108. The USB box 308 is detachably-placed on the bottom surface 1140 and can be positioned to receive wireless signals from the fire and smoke alarm device 104.

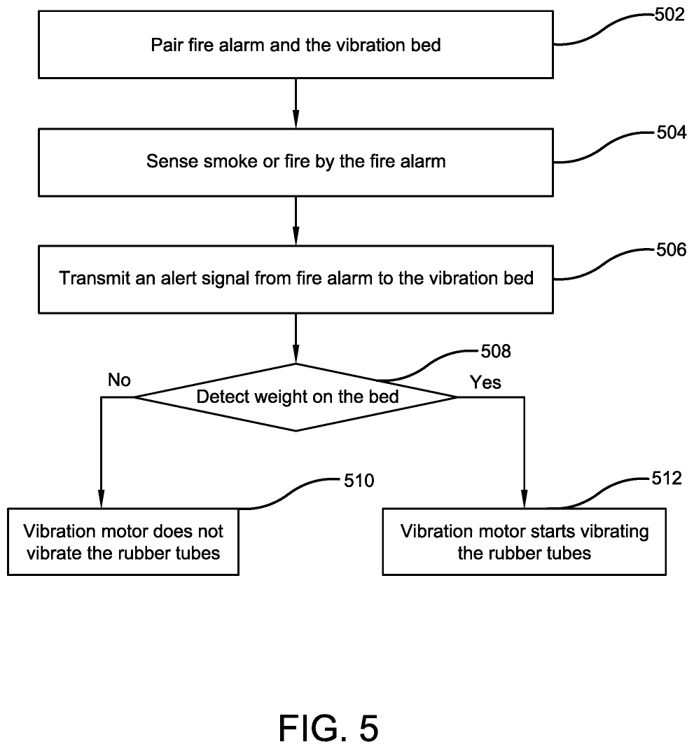

FIG. 5 illustrates a flow diagram showing exemplary steps in generating vibration in the vibrating bed for waking up user(s) lying on the bed in accordance with the disclosed architecture. Initially, the fire and smoke alarm 104 and the vibrating bed 102 are paired or connected together using the wireless communication channel such as Bluetooth (Block 502). Then, smoke or fire is sensed by the fire and smoke alarm (Block 504) and when the fire or smoke is detected by the alarm device, a wireless alert signal is transmitted from the fire or smoke alarm to the vibrating bed (Block 506). The weight sensor detects the weight (such as of a user) on the bed (Block 508) and if weight is detected, then the process moves to Block 512 where the vibration motor starts shaking and vibrating the rubber tubes to vibrate the user for waking. In case the weight is not detected by the weight sensor, then, the vibration motor is not activated (Block 510).

FIG. 6 illustrates a flow diagram showing steps performed in increasing the frequency of vibration produced by the vibrating motor used in the vibrating bed 102 of the present invention in accordance with the disclosed architecture. As shown, initially the vibration motor starts vibrating the rubber tubes (Block 602) and a time value is detected for which the vibration has been performed wherein the vibration time is also compared to a preconfigured threshold (Block 604). Then, weight on the bed is checked (Block 606) and in case the weight is detected, vibrations produced by the vibration motor in the rubber tubes are increased (Block 608) and in case weight is not detected, then, the vibrations are stopped (Block 610).

By using weight determination or detection, the method detects if a person is still lying on the bed even after the bed has been vibrated for a predetermined time period due to smoke/fire detection. In case a person is lying on the bed, even after the bed is vibrated for a predetermined time period, the method helps in increasing the vibration level to the bed, and therefore, increases the alarm level to wake up the person and thereby assist in saving the person from residential fire.

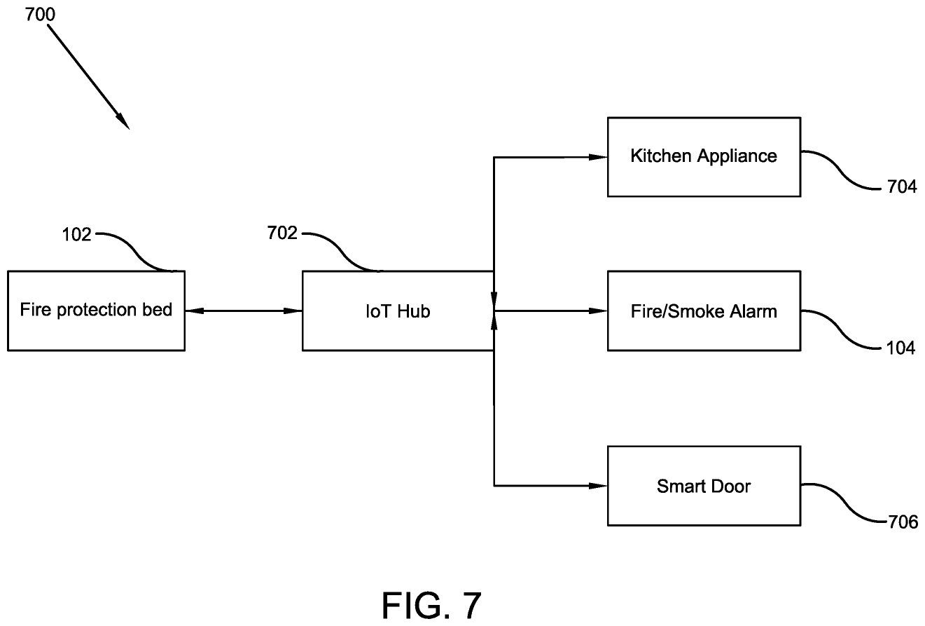

FIG. 7 illustrates another schematic view of the fire and smoke prevention system of the present invention with the fire protection bed 102 is in communication with an Internet of Things (IoT) hub 702 in accordance with the disclosed architecture. An advantage of the vibrating bed 102 of the present invention is that it can be communicatively coupled with an IoT hub 702 using the wireless module and then can become a part of a smart home or IoT network. The bed 102 can communicate with the IoT network using any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network can be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Bluetooth low energy (BLE), Internet Protocol (IP) data casting, satellite, mobile ad-hoc network (MANET), and the like, or any combination thereof.

The IoT hub 702 is further communicatively coupled to a variety of smart devices such as a smart kitchen appliance 704, the fire/smoke alarm 104, smart door 706 and many more. In case the IoT hub 702 receives an alert signal from any of these smart devices, the IoT hub 702 can transmit a wireless instruction signal to the bed 102 to actuate the vibrating motor and starts vibrating the bed 102. As an example, the smart door 706 can alert the IoT hub 702 in case the door opens during the night, after 1 AM for example. In such a case, the IoT hub 702 can alert the bed 102 to vibrate, thereby causing the user to wake up in order to prevent any burglary. It should be appreciated that direct communication between any of the smart devices and the vibrating bed 102 is also possible in a manner similar to the schematic shown in FIG. 1 of the present invention.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “fire and smoke prevention system”, “fire and smoke alert system”, and “system” are interchangeable and refer to the fire and smoke prevention system 100,700 of the present invention. Also, as used herein “vibrating bed”, “vibration bed”, “fire protection bed” and “bed” are interchangeable and refer to the vibrating bed 102 of the present invention.

Notwithstanding the forgoing, the fire and smoke prevention system 100,700 and the vibrating bed 102 of the present invention can be of any suitable size and configuration as is known in the art without affecting the overall concept of the invention, provided that it accomplishes the above-stated objectives. One of ordinary skill in the art will appreciate that the size, configuration, and material of the fire and smoke prevention system 100,700 and the vibrating bed 102 as shown in the FIGS. are for illustrative purposes only, and that many other sizes and shapes of the fire and smoke prevention system 100,700 and the vibrating bed 102 are well within the scope of the present disclosure. Although the dimensions of the fire and smoke prevention system 100,700 and the vibrating bed 102 are important design parameters for user convenience, the fire and smoke prevention system 100,700 and the vibrating bed 102 can be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A fire and smoke alert system comprising:
a vibrating bed including a wireless communication with a room fire and smoke alarm device;
wherein said vibrating bed includes a processor, a vibrating motor, and a mattress vibrating mechanism;
wherein said processor actuates said vibrating motor upon receiving an alarm signal from said room fire and smoke alarm device; and
wherein said vibrating motor initiates said mattress vibrating mechanism.

2. The fire and smoke alert system of claim 1, wherein said mattress vibrating mechanism includes vibrating rubber tubes.

3. The fire and smoke alert system of claim 2, wherein said vibrating bed includes a speaker for sounding an audible alarm when said processor receives said alarm signal from said room fire and smoke alarm device.

4. The fire and smoke alert system of claim 3, wherein said room fire and smoke alarm device includes a speaker for sounding an audible alarm upon receiving an alarm signal from said room fire and smoke alarm device.

5. The fire and smoke alert system of claim 3, wherein said processor includes a weight sensor for detecting a weight on said vibrating bed, and further wherein said processor increases a frequency of said vibrating motor while said weight is detected by said weight sensor.

6. The fire and smoke alert system of claim 4, wherein said processor includes an actuation button for manual operation of said processor.

7. The fire and smoke alert system of claim 4, wherein said room fire and smoke alarm device includes an LED for actuating upon receiving an alarm signal from said room fire and smoke alarm device.

8. The fire and smoke alert system of claim 4, wherein said wireless communication is selected from a group consisting of a local area network (LAN), a metropolitan area network (MAN), a wide area network (WAN), a public data network, and a short range wireless network.

9. The fire and smoke alert system of claim 4, wherein said wireless communication is a network selected from a group consisting of an enhanced data rates for global evolution (EDGE), a general packet radio service (GPRS), a global system for mobile communications (GSM), an Internet protocol multimedia subsystem (IMS), a universal mobile telecommunications system (UMTS), a worldwide interoperability for microwave access (WiMAX), a Long Term Evolution (LTE) network, a code division multiple access (CDMA), a wideband code division multiple access (WCDMA), a wireless fidelity (WiFi), a wireless LAN (WLAN), a Bluetooth®, and a mobile ad-hoc network (MANET).

10. A fire and smoke alert system comprising:
a vibrating bed including a wireless communication with a room fire and smoke alarm device;
wherein said vibrating bed includes a processor, a vibrating motor, and a mattress vibrating mechanism;
wherein said processor includes a weight sensor for detecting a weight on said vibrating bed;
wherein said processor actuates said vibrating motor upon receiving an alarm signal from said room fire and smoke alarm device and upon detecting said weight from said weight sensor; and
wherein said vibrating motor initiates said mattress vibrating mechanism.

11. The fire and smoke alert system of claim 10, wherein said mattress vibrating mechanism includes vibrating rubber tubes.

12. The fire and smoke alert system of claim 10, wherein said vibrating bed includes a speaker for sounding an audible alarm when said processor receives said alarm signal from said room fire and smoke alarm device.

13. The fire and smoke alert system of claim 12, wherein said room fire and smoke alarm device includes a speaker for sounding an audible alarm upon receiving an alarm signal from said room fire and smoke alarm device.

14. The fire and smoke alert system of claim 10, wherein said processor includes an actuation button for manual operation of said processor.

15. The fire and smoke alert system of claim 13, wherein said room fire and smoke alarm device includes an LED for actuating upon receiving an alarm signal from said room fire and smoke alarm device.

16. The fire and smoke alert system of claim 10, wherein said wireless communication is selected from a group consisting of a local area network (LAN), a metropolitan area network (MAN), a wide area network (WAN), a public data network, and a short range wireless network.

17. The fire and smoke alert system of claim 10, wherein said wireless communication is a network selected from a group consisting of an enhanced data rates for global evolution (EDGE), a general packet radio service (GPRS), a global system for mobile communications (GSM), an Internet protocol multimedia subsystem (IMS), a universal mobile telecommunications system (UMTS), a worldwide interoperability for microwave access (WiMAX), a Long Term Evolution (LTE) network, a code division multiple access (CDMA), a wideband code division multiple access (WCDMA), a wireless fidelity (WiFi), a wireless LAN (WLAN), a Bluetooth®, and a mobile ad-hoc network (MANET).

18. An emergency alert system comprising:
a vibrating bed including a wireless communication with a room fire and smoke alarm device and a smart door device;
wherein said vibrating bed includes a processor, a vibrating motor, and a mattress vibrating mechanism;
wherein said processor actuates said vibrating motor upon receiving an alarm signal from said room fire and smoke alarm device or upon receiving an alarm signal from said smart door device; and
wherein said vibrating motor initiates said mattress vibrating mechanism.

19. The fire and smoke alert system of claim 18, wherein said wireless communication is selected from a group consisting of a local area network (LAN), a metropolitan area network (MAN), a wide area network (WAN), a public data network, and a short range wireless network.

20. The fire and smoke alert system of claim 18, wherein said wireless communication is a network selected from a group consisting of an enhanced data rates for global evolution (EDGE), a general packet radio service (GPRS), a global system for mobile communications (GSM), an Internet protocol multimedia subsystem (IMS), a universal mobile telecommunications system (UMTS), a worldwide interoperability for microwave access (WiMAX), a Long Term Evolution (LTE) network, a code division multiple access (CDMA), a wideband code division multiple access (WCDMA), a wireless fidelity (WiFi), a wireless LAN (WLAN), a Bluetooth®, and a mobile ad-hoc network (MANET).
